(19) United States
(12) Patent Application Publication
(10) Pub. No.: US 2022/0040263 A1
(43) Pub. Date: Feb. 10, 2022

(54) COMPANION DIAGNOSTIC FOR COMBINATION LENALIDOMIDE AND ERYTHROPOIETIN TREATMENT

(71) Applicant: H. Lee Moffitt Cancer Center and Research Institute, Inc., Tampa, FL (US)

(72) Inventor: Alan F. List, Tampa, FL (US)

(21) Appl. No.: 17/189,664

(22) Filed: Mar. 2, 2021

Related U.S. Application Data
(63) Continuation of application No. 16/318,512, filed on Jan. 17, 2019, now abandoned.
(60) Provisional application No. 62/370,989, filed on Aug. 4, 2016.

(51) Int. Cl. A61K 38/18; A61K 31/454; G01N 33/569; G01N 33/574; A61P 7/06; C12Q 1/6883

(52) U.S. Cl. CPC A61K 38/1816; A61K 31/454; G01N 33/56966; G01N 33/57407; A61P 7/06; G01N 2333/70582; G01N 2333/70589; G01N 2800/52; C12Q 2600/106; C12Q 2600/158; C12Q 1/6883

(57) ABSTRACT

Disclosed herein is a companion diagnostic to predict efficacy of combination lenalidomide and erythropoietin treatment in patients with a erythropoietin (Epo)-refractory, Lower Risk (LR) Non-deletion 5q [Del(5q)] myelodysplastic syndrome (MDS). The method involves assaying erythroid precursors from a biological sample from the subject for a CD45 isoform profile, and treating the subject with a combination of lenalidomide and erythropoietin if the erythroid precursors have a predominance of large CD45RA and CD45RB isoforms compared to small CD45RO isoform.

### CROSS-REFERENCE TO RELATED APPLICATIONS

This application is a continuation of copending application Ser. No. 16/318,512, filed Jan. 17, 2019, which is a National Stage of International Application No. PCT/US2017/045527, filed Aug. 4, 2017, which claims benefit of U.S. Provisional Application No. 62/370,989, filed Aug. 4, 2016, which is hereby incorporated herein by reference in its entirety.

### STATEMENT REGARDING FEDERALLY SPONSORED RESEARCH OR DEVELOPMENT

This invention was made with Government Support under Grant No. CA131076 awarded by the National Institutes of Health. The Government has certain rights in the invention.

### BACKGROUND

Myelodysplastic syndromes (MDS) are hematopoietic stem cell malignancies characterized by dysplastic and ineffective hematopoiesis. MDS bone marrow precursors have a larger cell size, deregulated proliferation and maturation, and accelerated attrition by programmed cell death.

Lenalidomide (Revlimid®, Celgene Corporation, NJ, USA) (LEN) was initially intended as a treatment for multiple myeloma, but has also shown efficacy in MDS. Lenalidomide has significantly improved overall survival in myeloma (which generally carries a poor prognosis). Lenalidomide is undergoing clinical trial as a treatment for Hodgkin's lymphoma, as well as non-Hodgkin's lymphoma, chronic lymphocytic leukemia and solid tumor cancers, such as carcinoma of the pancreas. Patients with an interstitial deletion of Chromosome 5q have a high rate of response to lenalidomide, but most MDS patients lack this deletion. Approximately 25% of patients without 5q deletions also benefit from lenalidomide therapy, but response in these patients is difficult to predict.

Ineffective erythropoiesis manifested as symptomatic anemia remains the principal therapeutic challenge for patients with lower risk myelodysplastic syndrome (MDS). Treatment with recombinant human erythropoietin (rhu-EPO) may improve red blood cell (RBC) production in select patients with low transfusion burden or low endogenous EPO production, however, the majority of patients are growth factor refractory with forced reliance upon RBC transfusions and its attendant risk of iron overload. Laboratory investigations have shown that progenitor clonogenic response to EPO is attenuated in MDS and is associated with impaired EPO-receptor (R) activation of its primary transcriptional target, Signal Transduction and Activator of Transcription (STAT)-5.

### SUMMARY

As disclosed herein, lenalidomide (CC-5013; Revlimid®), has significant erythropoietic activity in lower risk MDS patients who have failed treatment with rhu-EPO. Results of three phase II clinical trials indicate that response to lenalidomide is karyotype-dependent, with a high frequency erythroid response (76%) and transfusion-independence (67%) that directly correlates with cytogenetic response (74%) in patients with the chromosome 5q31 interstitial deletion (del5q); whereas in patients with normal or alternate karyotypes, the MER rate is lower (25-30%) with infrequent cytogenetic responses and persistence of dysplasia in responding patients. These observations suggest that the hematologic effects of lenalidomide in MDS derive from interaction with discrete targets that are karyotype-dependent, i.e., (1) the selective suppression of del5q clones, and (2) potentiation of erythropoiesis in non-del5q erythroid precursors.

As disclosed herein, lenalidomide enhances EPO-induced STAT5 activation in non-del5q erythroid precursors, whereas in 5q31-deleted cells, it promotes programmed cell death. Furthermore, lenalidomide is a protein tyrosine phosphatase (PTP) inhibitor that relieves suppression of the EPO-R signal in non-del5q cells, while inducing cell cycle arrest and apoptosis in del5q cells by inhibiting a key haplo-deficient phosphatase encoded at chromosome 5q31 regulating G2/M transition. Without wishing to be bound by theory, lenalidomide may restore effective erythropoiesis in MDS by dual mechanisms that are karyotype-dependent: (a) the potentiation of EPO-R activation of STAT5 in non-del5q MDS through relief of signal inhibition by the CD45 PTP, and (b) selective cytotoxicity to del5q31 clones through the inhibition of critical haplo-deficient regulatory phosphatases, including the cell division cycle-25C (CDC25C) dual specificity phosphatase.

Lenalidomide restores sensitivity to rhEpo in Epo-refractory LR-non-del(5q) MDS patients to yield durable and significantly higher rates of erythroid response to combination treatment without added toxicity. Erythroid CD45 isoform profile is shown herein to serve as a response biomarker for selection of candidates for this combination therapy.

Therefore, disclosed herein is a companion diagnostic to predict efficacy of combination lenalidomide and erythropoietin treatment in patients with a erythropoietin (Epo)-refractory, Lower Risk (LR) Non-deletion 5q [Del(5q)] myelodysplastic syndrome (MDS).

Disclosed herein is a method for predicting whether a patient with a erythropoietin (Epo)-refractory, Lower Risk (LR) Non-deletion 5q [Del(5q)] myelodysplastic syndrome (MDS) will respond to treatment with lenalidomide and erythropoietin. The method involves assaying CD71^{Hi} erythroid precursors from a biological sample from a patient in need thereof for a CD45 isoform profile, wherein a predominance of small CD45 RO isoform compared to large CD45 RA and CD45 RB isoforms is an indication that the patient will respond to treatment with lenalidomide and erythropoietin.

Also disclosed is a method for treating a patient with a erythropoietin (Epo)-refractory, Lower Risk (LR) Non-deletion 5q [Del(5q)] myelodysplastic syndrome (MDS), comprising assaying CD71^{Hi} erythroid precursors from a biological sample from a patient in need thereof for a CD45 isoform profile, and treating the patient with a combination of lenalidomide and erythropoietin if the erythroid precursors have a predominance of small CD45 RO isoform compared to large CD45 RA and CD45 RB isoforms. The method involves assaying erythroid precursors from a biological sample from the subject for a CD45 isoform profile, and treating the subject with a combination of lenalidomide and erythropoietin if the erythroid precursors have a predominance of large CD45RA and CD45RB isoforms compared to small CD45RO isoform.

In some embodiments, the biological sample is assayed for CD45 isoforms on CD71^{Hi} erythroid precursors by flow cytometry. In some embodiments, the biological sample is assayed for gene expression of CD45 isoforms. In some embodiments, the biological sample comprises bone marrow mononuclear cells (BM-MNC).

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A and 1B show Lenalidomide potentiates clonogenic response to lenalidomide (CC-5013). FIG. 1A shows 2×10⁵ MDS BM-MNC pretreated with CC-5013 in IMDM+10% FBS at 37° C. for 2 hours, then washed with PBS and plated in triplicate in Cytokine Defined media (100 U/ml GMCSF, 100 U/ml IL-3, and 5 ng/ml SCF; R & D Systems, Minneapolis, Minn.) in methylcellulose+30% FBS with 3 U/ml EPO and incubated in a moist atmosphere with 5% CO₂. Colonies were scored after 14 days incubation and the growth of bone marrow colony-forming unit granulocyte, erythroid, macrophage, megakaryocyte (CFU-GEMM); burst-forming unit-erythroid (BFU-E); and colony-forming unit granulocyte-macrophage (CFU-GM) assessed. FIG. 1B shows UT-7 cells treated with CC-5013 for two hours in alpha-MEM+0.4% BSA at 37° C., then washed with PBS. Cells were then plated in methylcellulose+10% FBS, 2000 cells/well, with or without erythropoietin (EPO). Colonies were scored at day seven.

FIGS. 2A and 2B show potentiation of STAT 5 activation by lenalidomide. FIG. 2A shows 10×10⁶ UT7 cells treated either with media and vehicle alone (control), CC5013 (2 hours), EPO for the time period indicated, or CC5013 followed addition of EPO for the time periods indicated. Whole cell lysates were assayed for actin (Sigma), STAT5 and phospho-STAT5 (Cell Signaling) by Western immunoblot as previously described. FIG. 2B shows nuclear extracts prepared from UT7 cells and incubated with the 32 P-labeled MGFe oligonucleotide probes, which binds STAT5 and then analyzed by EMSA for STAT5 DNA binding. K562 leukemia cells served as a positive control.

FIG. 3 shows change in EPO-induced phospho-STAT5 mean fluorescence Intensity (MFI) in CD71+ primary MDS cells with lenalidomide treatment. BM-MNC were serum deprived in IMDM and 0.4% BSA for 1 hour, then treated as described in FIG. 5. RHu-EPO stimulated cells were treated 5 U/ml×15 minutes at 37° C. Cells were fixed in BD Cytofix (Beckon Dickenson) ×10 minutes, washed in BD Stain Buffer then re-suspended in ice cold 90% methanol. After washing ×2, cells were stained with anti-pSTAT5-phycoerthryin (PE) conjugated antibody and CD71-allophycocyanin (APC) conjugated antibody [20 μg/10⁶ cells]. Cells were washed ×2 with stain buffer before transfer to FACScan tubes and analysis on the FACScan flow cytometer. The gated CD71+ cell population accounted for 11.35% of BM-MNC. Results reflect phospho-STAT5:PE MFI in a representative patient specimen. Anti-pSTAT5, CD71-APC and CD34-APC antibodies were purchased from BD-Pharmingen.

FIG. 4 shows lenalidomide (CC5013) potentiation of EPO-STAT5 phosphorylation is Jak2 and Src-dependent. 10×10⁷ UT7 cells were serum starved then treated with SKI 606 or AG490 for 30 minutes, CC5013 for 2 hours, and stimulated with EPO (1 U/ml) for 10 minutes at 37° C. Cells were lysed and 15 mg run on a 4-15% Gradient gel (Bio-rad). Antibodies included p-STAT5 (Upstate, 1:1000 in 5% TBS/milk), total STAT5 (Cell Signaling, 1:10000 in 5% TBS/milk), and p-Jak2 (Santa Cruz, 1:500 in 5% TBS/milk). Blots were developed with an ECL detection kit (Amersham).

FIGS. 5A and 5B show lenalidomide potentiates c-Src activation. FIG. 5A shows UT7 cells treated with either vehicle alone (control), CC5013 (2 hours), EPO (30 minutes), or CC5013 followed by addition of EPO at the concentrations indicated. Cells were lysed and assayed for β-actin (Sigma), or Src, p-Src416, p-Src 527 and Bcl-XL (Cell Signaling) by Western immunoblot. Protein loading was normalized to β-actin. Relative Src activation in FIG. 5B represents the ratio of p-Src416 (activation site phosphorylation) to p-Src527 (C-terminal inhibitory p-tyrosine).

FIG. 6 shows lenalidomide suppresses Csk and decreases Tyr527 phosphorylation. Serum starved UT7 cells were treated with either vehicle alone (control), CC5013 (2 hours), EPO (30 minutes), or CC5013 followed by addition of EPO at the concentrations indicated. Cells were lysed and assayed for β-actin (Sigma), phospho-Lyn507, total Lyn and Csk (Cell Signaling) by Western immunoblot as described previously. Protein loading was normalized to β-actin, and immunoblot signals of phosphorylated Lyn were normalized to total Lyn and actin by band densitometry.

FIG. 7 shows lenalidomide (CC-5013) inhibits CD45 PTP activity. CD45-specific PTP activity was assayed as outlined in the BIOMOL Green™ CD45 Tyrosine Phosphatase Assay Kit. Briefly, 75 U of rhu-CD45 (Calbiochem) was incubated for 1H at 37° C. with the indicated inhibitors (1 μM CC5013 and thalidomide, 100 μM Na Orthovanadate, and 50 μM RWJ-60475 (CD45-specific PTP inhibitor) in 1× assay buffer. The substrate pp60c-Src was added at 200 μM and incubated for 1H at 37° C. Samples were read on a plate reader at 620 nm. Percent PTP activity was calculated according to the equation (OD of test sample/OD of control)×100.

FIGS. 8A and 8B shows apoptosis induction by lenalidomide is specific for 5q-deleted cells. AML-EH5q (FIG. 8A) and U937 (FIG. 8B) cells were exposed to lenalidomide, thalidomide or vehicle at the concentrations indicated for 24 hours in IMDM and 20% FBS and apoptosis assessed by flow cytometry using Annexin-V/PI staining. Briefly, cells are incubated for 20 minutes at room temperature in 50 mM HEPES (pH 7.4) and 150 mM NaCl containing 2 mM calcium chloride with a mixture of 1 μg/mLAnnexin V-FITC (Boehringer Mannhein, Mannhein, Germany) and 2.5 μg/mL propidium iodide (PI; Sigma-Aldrich) before analysis. Lenalidomide selectively induced cell death in AML-EH5q cells in a concentration-dependent fashion whereas thalidomide was comparatively inactive in both cell lines tested.

FIG. 9 depicts lenalidomide docked into the homology model of Cdc25C.

FIG. 10 shows lenalidomide (CC-5013) inhibits CDC25C PTP activity. 200 U of rhu-CDC25C (BIOMOL) was incubated in assay buffer (100 mM Tris-HCL, pH 8.2, 40 mM NaCl, 1 mM DTT, 0.1% BSA, and 20% glycerol) for 1 hour at 37 C with the indicated inhibitors CC-5013, Thalidomide, and CDC25 Phosphatase Inhibitor, NSC, Calbiochem) at the concentrations indicated. The substrate OMFP (3-0-Methylfluorescein phosphate cyclohexylammonium salt, Sigma) was added at 500 μM and tubes were incubated for 60 minutes at 37° C. Samples were read on a spectrophotometer at 477 nm. Percent activity was calculated by subtracting background absorbance and dividing each sample by the CDC25C+control.

FIG. 11 shows a gating strategy and quantitation of CD45 isoform ratios. Representative flow cytometry diagrams of bone marrow mononuclear cells (BM-MNC) that were stained using CD71:APC, CD45RA:FITC, CD45RB:PE, and CD45RO:PE-CY5 antibodies. Erythroid progenitors (CD71+ cells) were gated and percentages of erythroid progenitors positive for each isoform (RA, RB, and RO) were calculated. Positive cells are illustrated by boxes and determined using the FMO (fluorescence minus one) method. Statistical analysis of CD45 isoforms was calculated as the log_10(CD45RA+CD45RB):CD45RO. Progenitors with a high ratio (top) were associated with lower response rates, whereas progenitors with low isoform ratios (bottom) were associated with higher response rates (p=0.04).

### DETAILED DESCRIPTION

Disclosed is a method for treating a patient with a erythropoietin (Epo)-refractory, Lower Risk (LR) Non-deletion 5q [Del(5q)] myelodysplastic syndrome (MDS) that involves assaying a biological sample from the subject for an erythroid CD45 isoform profile, and treating the subject with a combination of lenalidomide and erythropoietin if the subject has a predominance of large RA and RB CD45 isoforms with primed PTP activity.

CD45 Isoforms

CD45 antigen, also known as protein tyrosine phosphatase, receptor type, C (PTPRC), is an enzyme that, in humans, is encoded by the PTPRC gene. The protein encoded by this gene is a member of the protein tyrosine phosphatase (PTP) family. PTPs are known to be signaling molecules that regulate a variety of cellular processes including cell growth, differentiation, mitotic cycle, and oncogenic transformation. This PTP contains an extracellular domain, a single transmembrane segment and two tandem intracytoplasmic catalytic domains, and thus belongs to receptor type PTP. This gene is specifically expressed in hematopoietic cells. This PTP has been shown to be an essential regulator of T- and B-cell antigen receptor signaling. It functions through either direct interaction with components of the antigen receptor complexes or by activating various Src family kinases required for the antigen receptor signaling.

The CD45 family consists of multiple members that are all products of a single complex gene. This gene contains 34 exons, and three exons of the primary transcripts are alternatively spliced to generate up to eight different mature mRNAs and after translation eight different protein products. These three exons generate the RA, RB and RC isoforms. Various other isoforms of CD45 exist: CD45RAB, CD45RAC, CD45RBC, CD45RO, CD45R (ABC). CD45RA is located on naive T cells and CD45RO is located on memory T cells. CD45 is also highly glycosylated. CD45R is the longest protein and migrates at 200 kDa when isolated from T cells. B cells also express CD45R with heavier glycosylation, bringing the molecular weight to 220 kDa, hence the name B220; B cell isoform of 220 kDa. B220 expression is not restricted to B cells and can also be expressed on activated T cells, on a subset of dendritic cells and other antigen-presenting cells.

Naive T lymphocytes express large CD45 isoforms and are usually positive for CD45RA. Activated and memory T lymphocytes express the shortest CD45 isoform, CD45RO, which lacks RA, RB, and RC exons. This shortest isoform facilitates T cell activation.

Many types and formats of immunoassays are known and all are suitable for detecting the disclosed CD45 isoforms. Examples of immunoassays are enzyme linked immunosorbent assays (ELISAs), radioimmunoassays (RIA), radioimmune precipitation assays (RIPA), immunobead capture assays, Western blotting, dot blotting, gel-shift assays, Flow cytometry, protein arrays, multiplexed bead arrays, magnetic capture, in vivo imaging, fluorescence resonance energy transfer (FRET), and fluorescence recovery/localization after photobleaching (FRAP/FLAP). In some embodiments, the method involves flow cytometric analysis of CD71^{Hi} erythroid cells for CD45 isoforms. CD45 exon-specific (e.g., RA, RB, RC and RO) antibodies are commercially available and can be used to detect and quantify CD45 isoform distribution on CD71^{Hi} erythroid cells.

Many types and formats of gene expression assays are known and all are suitable for detecting the disclosed CD45 isoforms as mRNA. A number of widely used procedures exist for detecting and determining the abundance of a particular mRNA in a total or poly(A) RNA sample. For example, specific mRNAs can be detected using Northern blot analysis, nuclease protection assays (NPA), in situ hybridization, or reverse transcription-polymerase chain reaction (RT-PCR).

As disclosed herein, lenalidomide can inhibit CD45 PTP activity and thereby restore STAT5 phosporylation after EPO stimulation. Also as disclosed herein, efficacy of lenalidomide and erythropoietin combination therapy can be predicted by predominance of large CD45 isoforms, which have primed PTP activity. Therefore erythroid CD45 isoform profile can discriminate response potential to EPO and lenalidomide treatment.

In some embodiments, the method involves determining the ratio of CD45RA and CD45RB isoforms to the small CD45RO isoform. Therefore, in some embodiments, the method involves treating the subject with a combination of lenalidomide and erythropoietin based on the subjects RA/RO ratio, RB/RO ratio, RA+RB/RO ratio, or a combination thereof.

For example, patients responding to EPO and lenalidomide can have a RA+RB:RO ratio of less than 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0.

In some embodiments, non-responders can have a RA+RB/RO ratio of at least 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, or 6.0.

Therefore, in some embodiments, the disclosed method involves treating the subject with a combination of lenalidomide and erythropoietin if the subject has a RA+RB:RO ratio less than 3.0, 2.9, 2.8, 2.7, 2.6, 2.5, 2.4, 2.3, 2.2, 2.1, or 2.0.

The disclosed combination of lenalidomide and erythropoietin can also be used in combination with any compound, moiety or group which has a cytotoxic or cytostatic effect. Drug moieties include chemotherapeutic agents, which may function as microtubulin inhibitors, mitosis inhibitors, topoisomerase inhibitors, or DNA intercalators, and particularly those which are used for cancer therapy.

The disclosed combination of lenalidomide and erythropoietin can be used in combination with a checkpoint inhibitor. The two known inhibitory checkpoint pathways involve signaling through the cytotoxic T-lymphocyte antigen-4 (CTLA-4) and programmed-death 1 (PD-1) receptors. These proteins are members of the CD28-B7 family of cosignaling molecules that play important roles throughout all stages of T cell function. The PD-1 receptor (also known as CD279) is expressed on the surface of activated T cells. Its ligands, PD-L1 (B7-H1; CD274) and PD-L2 (B7-DC; CD273), are expressed on the surface of APCs such as dendritic cells or macrophages. PD-L1 is the predominant ligand, while PD-L2 has a much more restricted expression pattern. When the ligands bind to PD-1, an inhibitory signal is transmitted into the T cell, which reduces cytokine production and suppresses T-cell proliferation. Checkpoint inhibitors include, but are not limited to antibodies that block PD-1 (Nivolumab (BMS-936558 or MDX1106), CT-011, MK-3475), PD-L1 (MDX-1105 (BMS-936559), MPDL3280A, MSB0010718C), PD-L2 (rHIgM12B7), CTLA-4 (Ipilimumab (MDX-010), Tremelimumab (CP-675,206)), IDO, B7-H3 (MGA271), B7-H4, TIM3, LAG-3 (BMS-986016).

Pharmaceutical Composition

Also disclosed is a pharmaceutical composition comprising lenalidomide and/or erythropoietin in a pharmaceutically acceptable carrier.

Recombinant human erythropoietin (rhEPO) can be produced by recombinant DNA technology in cell culture. Several different pharmaceutical agents are available with a variety of glycosylation patterns and are collectively called erythropoiesis-stimulating agents (ESA). Major examples are epoetin alfa and epoetin beta. Erythropoietins available for use as therapeutic agents are produced by recombinant DNAtechnology in cell culture, and include Epogen/Procrit (epoetin alfa) and Aranesp (darbepoetin alfa).

The drug compound having the adopted name “lenalidomide” has a chemical name 3-(4-amino-1-oxo-1,3-dihydro-2H-isoindol-2-yl)piperidine-2,6-dione, and is structurally represented by Formula I.

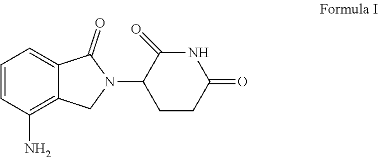 [SMILES: Nc1cccc2c1CN(C1CCC(=O)NC1=O)C2=O]

Lenalidomide was approved by the U.S. Food and Drug Administration on Dec. 27, 2005 for treating patients with low or intermediate-1 risk MDS with 5q-with or without additional cytogenetic abnormalities. The drug is commercially marketed in products sold by Celgene Corporation under the brand name REVLIMID™ in the form of capsules having the strengths 5 mg, 10 mg, 15 mg, and 25 mg. Muller et al., in U.S. Pat. No. 5,635,517 disclose substituted 1-oxo-2-(2,6-dioxopipehdin-3-yl) isoindolines derivatives, pharmaceutical compositions containing these compounds and their use in the treatment of cancer. It also discloses a process for the preparation of these compounds, which involves hydrogenation of a nitro group to an amino group, using palladium on carbon in 1,4-dioxane solvent. Muller et al., in U.S. Patent Application Publication No. 2006/0052609, disclose another process for the preparation of lenalidomide. The process involves the hydrogenation of (S)- or racemic 3-(4-nitro-1-oxo-1,3-dihydroisoindol-2-yl)-piperidine-2,6-dione using 10% palladium on carbon in methanol, to form (S)- or racemic 3-(4-amino-1-oxo-1,3-dihydro-2H-isoindol-2-yl)piperidine-2,6-dione. International Patent Application WO2009/114601 discloses a method for preparing substantially pure, and pure amorphous form of lenalidomide and solid dispersions containing amorphous lenalidomide.

Pharmaceutical carriers are known to those skilled in the art. These most typically would be standard carriers for administration of drugs to humans, including solutions such as sterile water, saline, and buffered solutions at physiological pH. For example, suitable carriers and their formulations are described in Remington: The Science and Practice of Pharmacy (21 ed.) ed. PP. Gerbino, Lippincott Williams & Wilkins, Philadelphia, Pa. 2005. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. The solution should be RNAse free. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Some of the compositions may be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

Methods of Treatment

The disclosed compositions, including pharmaceutical composition, may be administered in a number of ways depending on whether local or systemic treatment is desired, and on the area to be treated. For example, the disclosed compositions can be administered intravenously, intraperitoneally, intramuscularly, subcutaneously, intracavity, or transdermally. The compositions may be administered orally, parenterally (e.g., intravenously), by intramuscular injection, by intraperitoneal injection, transdermally, extracorporeally, ophthalmically, vaginally, rectally, intranasally, topically or the like, including topical intranasal administration or administration by inhalant.

Parenteral administration of the composition, if used, is generally characterized by injection. Injectables can be prepared in conventional forms, either as liquid solutions or suspensions, solid forms suitable for solution of suspension in liquid prior to injection, or as emulsions. A revised approach for parenteral administration involves use of a slow release or sustained release system such that a constant dosage is maintained.

The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein. For example, effective dosages and schedules for administering the compositions may be determined empirically, and making such determinations is within the skill in the art. The dosage ranges for the administration of the compositions are those large enough to produce the desired effect in which the symptoms disorder are affected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. A typical daily dosage of the disclosed composition used alone might range from about 1 μg/kg to up to 100 mg/kg of body weight or more per day, depending on the factors mentioned above.

In some embodiments, the molecule containing lenalidomide and/or erythropoietin is administered in a dose equivalent to parenteral administration of about 0.1 ng to about 100 g per kg of body weight, about 10 ng to about 50 g per kg of body weight, about 100 ng to about 1 g per kg of body weight, from about 1 μg to about 100 mg per kg of body weight, from about 1 μg to about 50 mg per kg of body weight, from about 1 mg to about 500 mg per kg of body weight; and from about 1 mg to about 50 mg per kg of body weight. Alternatively, the amount of molecule containing lenalidomide and/or erythropoietin administered to achieve a therapeutic effective dose is about 0.1 ng, 1 ng, 10 ng, 100 ng, 1 μg, 10 μg, 100 μg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 30 mg, 40 mg, 50 mg, 60 mg, 70 mg, 80 mg, 90 mg, 100 mg, 500 mg per kg of body weight or greater.

### Definitions

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

As used herein, “administering” an agent may be performed using any of the various methods or delivery systems well known to those skilled in the art. The administering can be performed, for example, orally, parenterally, intraperitoneally, intravenously, intraarterially, transdermally, sublingually, intramuscularly, rectally, transbuccally, intranasally, liposomally, via inhalation, vaginally, intraoccularly, via local delivery, subcutaneously, intraadiposally, intraarticularly, intrathecally, into a cerebral ventricle, intraventicularly, intratumorally, into cerebral parenchyma or intraparenchchymally. The following delivery systems, which employ a number of routinely used pharmaceutical carriers, may be used but are only representative of the many possible systems envisioned for administering compositions in accordance with the invention.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: Lenalidomide Potentiates EPO-Induced STAT5 Activation

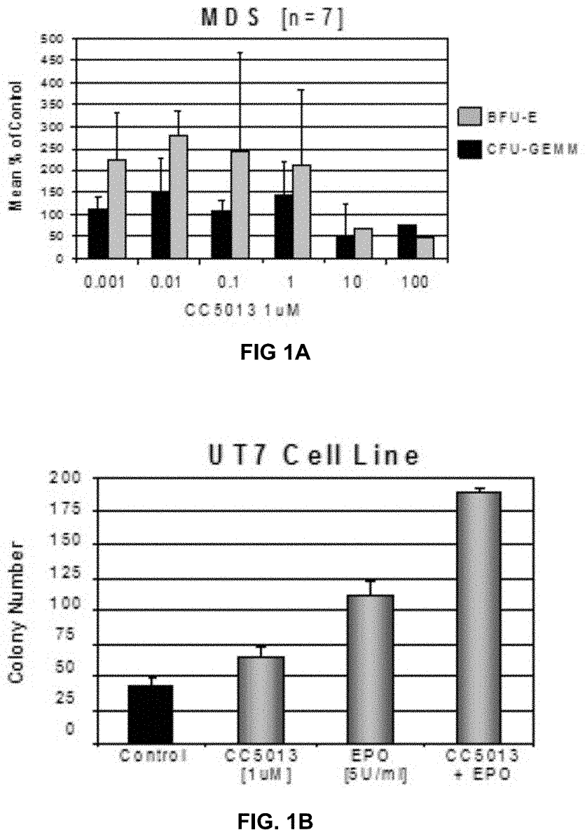

Clinical investigations show that lenalidomide enhances erythropoiesis in a significant proportion of patients with lower risk MDS. To investigate the mechanism by which lenalidomide promotes erythropoiesis, the effect of lenalidomide on erythroid progenitor colony forming capacity was evaluated in lower risk patients [FIG. 1A]. Two-hour exposure to lenalidomide prior to initiation of BM-MNC in methylcellulose culture enhanced BFU-E formation over a concentration range of 0.001-1.0 μM. Growth of multipotent progenitors (CFU-GEMM) and myeloid progenitors [CFU-GM] were unchanged indicating that the potentiating effect of lenalidomide is lineage and/or cytokine-dependent. Erythroid progenitor cell lines were next evaluated to identify a model for investigation of the mechanism of lenalidomide's erythroid promoting effect. The UT7 cell line showed a similar pattern of potentiation of EPO-induced CFC by lenalidomide to that in MDS BFU-E [FIG. 1B].

Treatment with lenalidomide [1 μM] alone had minimal effect on colony forming capacity, whereas clonogenic response to EPO increased colony recovery more than two-fold. When UT7 cells were exposed to lenalidomide prior to culture initiation with EPO, supra-additive colony forming capacity was observed that exceeded growth induced by EPO and/or lenalidomide treatments [P=0.001].

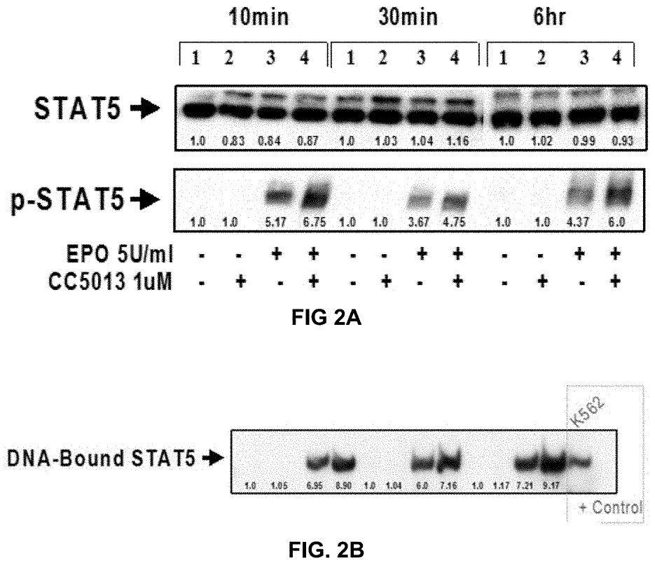

To determine if lenalidomide modulates EPO-R signaling to enhance cytokine induced clonogenic response, STAT5 phosphorylation and its nuclear trans-activation was evaluated after treatment with either lenalidomide or EPO alone, or the combination [FIG. 2A].

Western immunoblot showed that lenalidomide increased EPO-induced phosphorylation of STAT5, whereas lenalidomide alone had no effect on STAT5 phosphorylation. Enhancement of STAT5 nuclear translocation was confirmed by electrophoretic mobility gel shift assay (EMSA), which demonstrated greater DNA binding of the transcription factor after combined lenalidomide and EPO treatment [FIG. 2B]. Potentiation of STAT5 phosphorylation by lenalidomide is immediate and sustained, supporting modulation of enzymatic activity rather than transcriptional response. These findings indicate that lenalidomide increases STAT5 activation in response to receptor engagement by its ligand and is that lenalidomide's potentiating effect on erythroid clonogenic response is ligand-dependent.

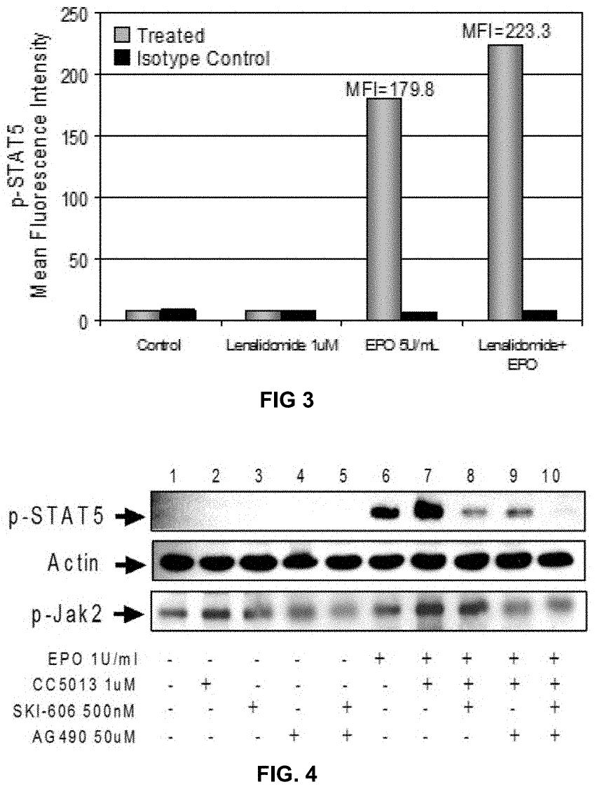

To confirm that lenalidomide enhances STAT5 phosphorylation in erythroid precursors from primary BM specimens, BM specimens were evaluated from Low- or Intermediate-1 risk MDS by flow cytometry. As in the UT7 cell line, lenalidomide increased EPO-induced phosphorylation of STAT5 in CD71+ erythroid precursors, whereas lenalidomide alone had no effect on STAT5 phosphorylation [FIG. 3]. The STAT5 phosphorylation delta (PD), i.e., the increase in mean fluorescence intensity (MFI) for EPO-induced STAT5 phosphorylation with the addition of lenalidomide compared to EPO alone was 43.5 relative MFI units, representing a 24% increase in pSTAT5 MF. These data confirm that UT7 cells are a reliable model for the study of EPO-R signal modulation by lenalidomide in erythroid precursors that is relevant to patient specimens.

### Example 2: Lenalidomide Potentiation of the EPO/STAT5 Signal is Mediated by Jak2 and SFK

To further evaluate the mechanism by which lenalidomide enhances the EPO-R signal, STAT5 activation was assessed in response to EPO+lenalidomide treatment using selective pharmacologic inhibitors of JAK (AG490) or Src (PD180970) kinases [FIG. 4].

Lenalidomide alone increased Jak2-phosphorylation [Lane 2] and further augmented EPO-induced Jak2-phosphorylation [Lane 7] that was abrogated by treatment with the Janus kinase inhibitor, AG490 [Lane 9] but not the Src kinase inhibitor, SKI-606 [Lane 8]. Both Src kinase inhibition with SKI-606 and Jak2 inhibition by AG490 suppress lenalidomide's potentiation of EPO-induced STAT5 phosphorylation. These findings indicate that potentiation of STAT5 activation by lenalidomide is mediated by Jak2 and Lyn/SFK, and that lenalidomide alone can relieve endogenous repression of Jak2 activity.

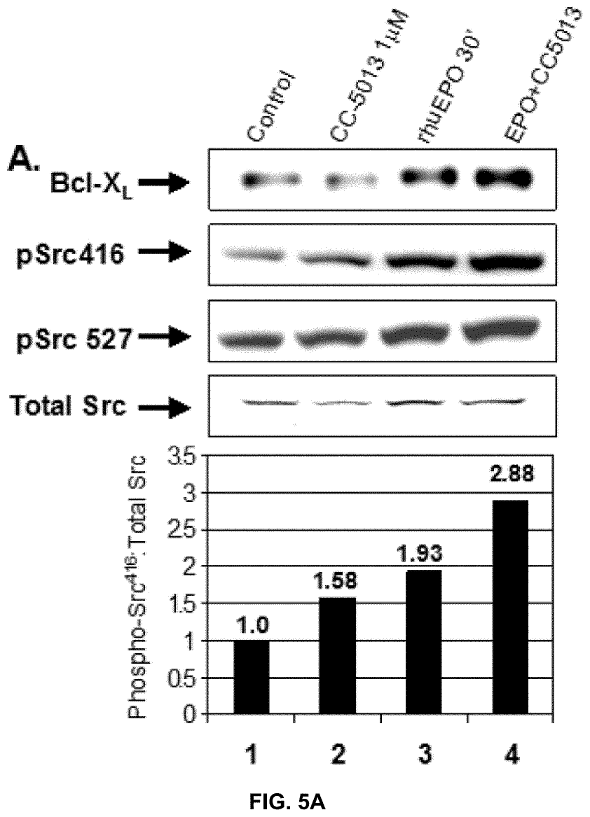

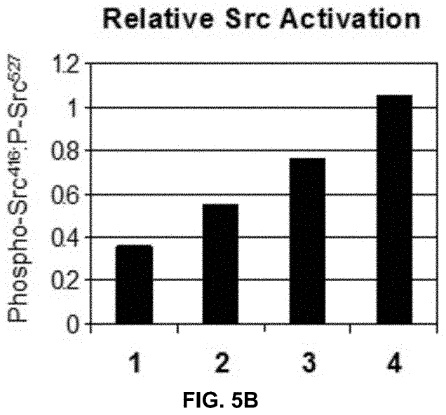

To confirm that EPO-R signal potentiation by lenalidomide is associated with activation of Src and Lyn kinases, Src activation was evaluated using phospho-tyrosine specific antibodies that recognize either the activation loop tyrosine (Tyr416) or the C-terminal inhibitory tyrosine (Tyr527) of c-Src. Lenalidomide alone increased phosphorylation of Tyr416 by 50% compared to the vehicle treated control [FIG. 5].

EPO further increased phosphorylation of Src Tyr416, however, when combined with lenalidomide, Src activation was supra-additive and increased nearly 3-fold compared to the vehicle control. Induction of the Bcl-2 family anti-apoptotic protein Bcl-XL was EPO-dependent, and was augmented by lenalidomide pre-treatment. Phosphorylation of the C-terminal inhibitory tyrosine (Tyr527) increased minimally, indicating that relative c-Src activation as measured by the ratio of phoso-Src416 to phoso-Src527 is increased in the presence of lenalidomide [FIG. 5B]. These findings suggest that lenalidomide enhances Epo-R/SFK signaling by relieving endogenous inhibition of Lyn/Src kinase signaling.

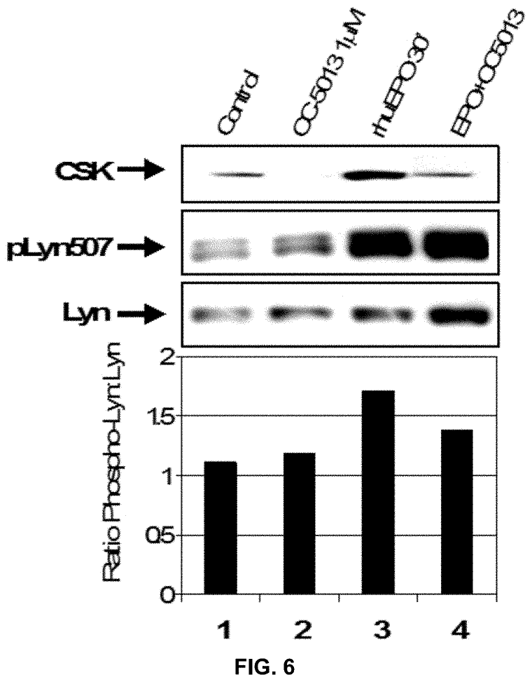

The Lyn/Src signal response is inhibited through both Csk phosphorylation of the C-terminal tyrosine residue enabled by SFK phosphorylation of the Csk-binding protein (Csk/Pag), and by the action of the CD45 PTP that dephosphorylatse the SFK activation loop tyrosine. There were minor changes in phosphorylation of the C-terminal tyrosine of Src and Lyn kinases despite significant increases in phosphorylation of the Src activation loop tyrosine [FIGS. 5 and 6]. A minor reduction in C-terminal tyrosyl phosphorylation relative to total Lyn was associated with a decrease in total Csk that was apparent with treatment with either lenalidomide alone or when combined with EPO [FIG. 6]. Total Lyn increased with sequential CC-5013/EPO treatment despite equal protein loading, suggesting that the relative reduction in C-terminal tyrosyl phosphorylation was sufficient to decrease Lyn's degradation. These findings suggest that enhancement of Src activation by lenalidomide is associated with suppression of Csk and interference with PTP dephosphorylation of the activation loop tyrosine through either suppression of CD45 or inhibition of phosphatase activity.

### Example 3: Lenalidomide Inhibits CD45 PTP Activity

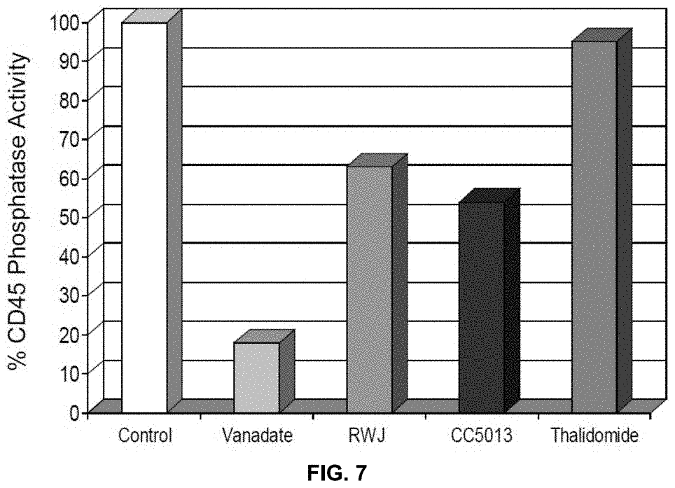

Observations that lenalidomide's potentiation of the EPO-R/STAT5 signal is immediate, and both Jak2 and Src/Lyn kinase dependent, suggests that lenalidomide may act by inhibiting the enzymatic activity of the CD45 PTP to enhance receptor signal response by both kinase pathways. This notion is in agreement with findings that lenalidomide suppressed cytosolic release of Csk that is dependent upon dephosphorylation of the Cbp/PAG Csk docking site (Tyr314) by CD45. To determine if lenalidomide inhibits CD45 phosphatase, recombinant CD45 PTP inhibition by lenalidomide and thalidomide was compared to the pan-PTP inhibitor sodium orthovanadate and the CD45-selective inhibitor, RWJ-60475 using the Biomol CD45 phosphatase assay. Thalidomide displays minimal inhibition of CD45 PTP activity, whereas lenalidomide is a potent inhibitor with nearly 50% inhibition at 1 μM, indicating a potency that exceeds that of the selective CD45 inhibitor, RWJ-60475. [FIG. 7].

### Example 4: Lenalidomide is Selectively Cytotoxic to Del5q Clones

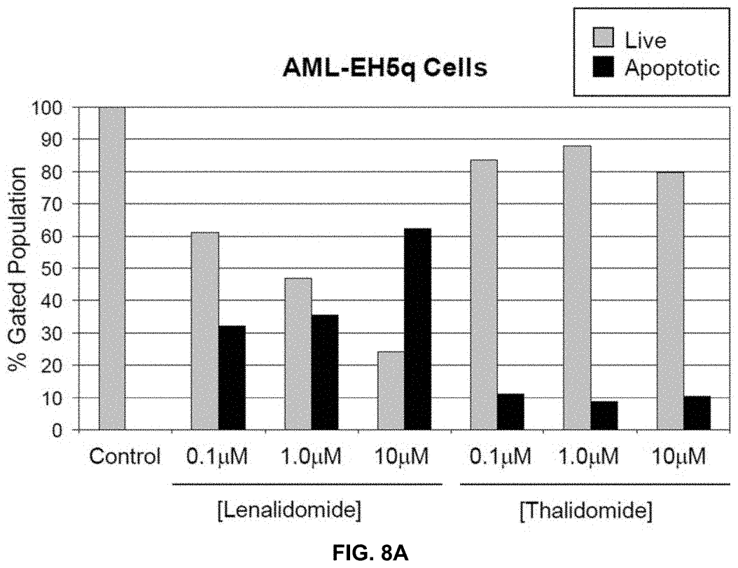

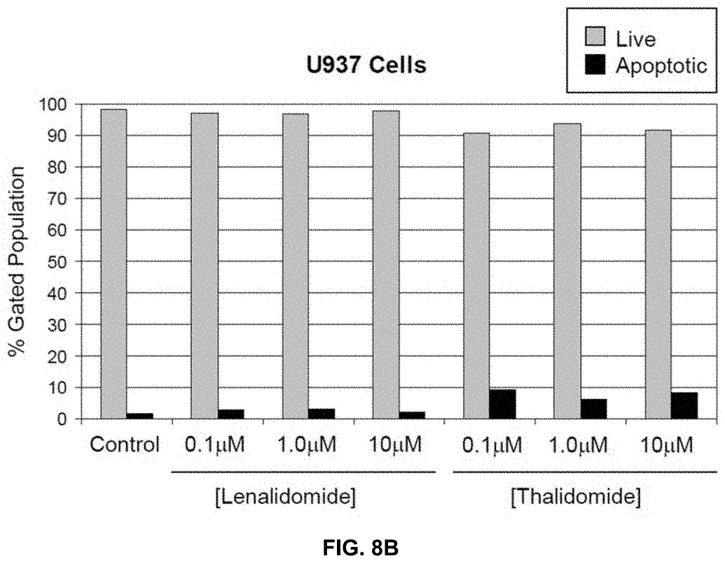

The hematologic effects of lenalidomide could derive from dual effects that are karyotype-dependent, i.e., (1) the potentiation of erythropoiesis in non-del5q erythroid precursors, and (2) selective suppression of del5q clones. To confirm that lenalidomide is selectively cytotoxic to del-5q clones, an AML cell line transformed from an MDS patient with isolated 5q31 deletion was cultivated and the cell line successfully maintained in suspension culture with recombinant myeloid growth factor supplementation. This cell line [designated AML-EH5q] is an AML cell line that harbors the isolated 5q-deletion. This cell line displays a karyotype of 46,XX, del (5)(q15q35) with a myeloid antigen phenotype characterized by strong expression of CD13, CD34 and CD117, and weak CD33, HLA-DR, CD11c, CD56 and CD45. To evaluate the selectivity of lenalidomide cytotoxic for del5q clones, apoptotic response to lenalidomide and thalidomide was compared in AML-EH5q and U937 (non-del5q) cells by flow cytometry. Findings indicate that lenalidomide is selectively cytotoxic to 5q-deleted cells, showing concentration dependent induction of programmed cell death in AML-EH5q cells whereas thalidomide is inactive at equimolar concentrations [FIG. 8]. Viability was unaffected at all concentrations for both agents in U937 cells. These findings support the hypothesis that lenalidomide selectively inhibits a critical target that is haplo-deficient in deletion 5q clones.

### Example 5: Lenalidomide Binds to and Inhibits the CDC25C Dual Specificity Protein Phosphatase

Findings that lenalidomide inhibits the CD45 PTP, suggest that selective cytotoxicity to del5q clones may be mediated by inhibition of a critical PTP that is encoded within the 1.5 Mb common deleted region (CDR) between 5q31-5q32, which is haplo-deficient in del5q clones. Examination of the CDR identified only one PTP, CDC25C, encoded on 5q31.2 that is a critical regulator of G2/M cell cycle transition. To determine if lenalidomide binds to the catalytic site of the CDC25C protein, a homology model of the phosphatase catalytic domain was first constructed for computational docking. The sequence of human Cdc25C was downloaded from the SwissProt database (entry P30307) and aligned with the sequence of the catalytic domain of human Cdc25B, the structure of which has been determined via X-ray crystallography (1QB0 in the protein databank once the catalytic domain of Cdc25C was located from this alignment. The sequence identity for the aligned catalytic domains is approximately 53% and the sequence homology is approximately 70%, which is sufficient to construct an accurate homology model. The SwissModel server was employed to build a homology model using the X-ray structure of Cdc25B (1QB0) as the template. The homology model was further processed using the Protein Preparation module of the First Discovery Suite (Schrödinger, Inc.). This computer program neutralizes surface residues, generates appropriate histidine tautomers, and performs constrained energy minimization to optimize the geometry of the model.

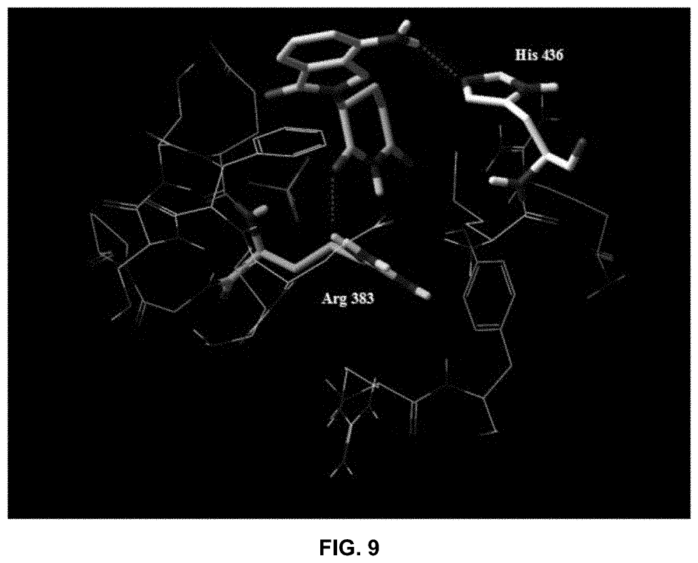

In preliminary studies, automated computational docking of lenalidomide, thalidomide and the Cdc25-specific inhibitor, NSC-663284, to the homology model was performed using the Glide 3.0 software (Schrödinger, Inc.). The docking grids were established relative to a bound sulfate ion positioned in the homology model in the same location as the one observed by X-ray crystallography in the active site of Cdc25B (1Qb0). In the best docking mode for lenalidomide and thalidomide, the piperidine-dione oxygens approximated positions to two of the oxygens of the sulfate anion described above and thus the piperidine-dione moiety presumably binds within the phosphate binding site. Unlike thalidomide, lenalidomide appears to be capable of forming a hydrogen bond to His 436 with the amino group on the drug's pthaloyl ring to reinforce binding. Both molecules appear capable of hydrogen bonding to Arg 383 as is the case for sulfate (and presumably phosphate). FIG. 9 illustrates the docking mode for lenalidomide. The corresponding docking scores for lenalidomide were stronger [−4.57 kcal/mol] compared and thalidomide [−4.06 kcal/mol. Similarly, when docked to the Cdc25C homology model, in the best docking mode, the quinoline-dione moiety in NSC-663284 was also bound to the presumed phosphate binding site with a docking score comparable to that for lenalidomide [−4.55 kcal/mol].

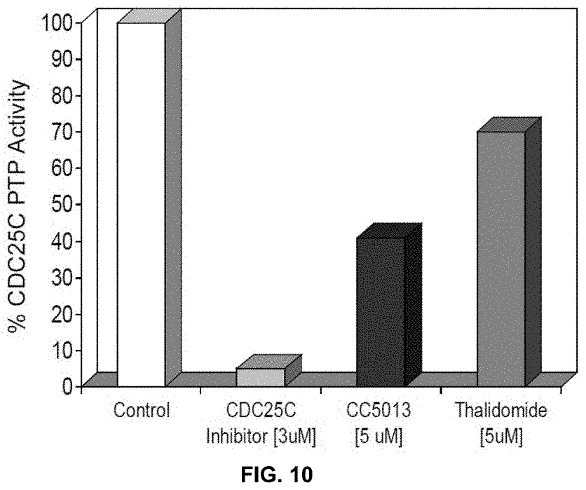

To determine if lenalidomide inhibits CDC25C phosphatase activity, rhu-CDC25C PTP inhibition by lenalidomide and thalidomide were compared to a CDC25C selective inhibitor using the 3-o-methylfluorescein phosphate (OMFP) hydrolysis assay [FIG. 10]. Findings indicate that lenalidomide is a potent and selective inhibitor of CDC25C. These data suggest that the haplo-deficient CDC25C is a target of lenalidomide in del-5q clones and therefore may account for selective cytotoxicity of the compound.

### Example 6: Combined Treatment with Lenalidomide (LEN) and Epoetin Alfa (EA) is Superior to Lenalidomide Alone in Patients with Erythropoietin (Epo)-Refractory, Lower Risk (LR) Non-Deletion 5q [Del(5q)] Myelodysplastic Syndromes (MDS)

Treatment with rhu-Epo ameliorates anemia in a subset of LR-MDS patients, however, effective salvage therapy is limited. LEN promotes erythroid lineage competence and expansion of primitive erythroid precursors in vitro. In the MDS-002 and MDS-005 trials, treatment with LEN improved erythropoiesis, yielding RBC transfusion-independence in 26% of azanucleoside-naive, transfusion-dependent (TD) LR, non-del(5q) MDS patients for a median of 10.2 and 7.75 months, respectively. LEN restores Epo-responsiveness in MDS progenitors by inducing the formation of lipid rafts enriched for signaling competent JAK2/Epo-receptor complexes with exclusion of large isoforms of the JAK2/lyn kinase-phosphatase CD45 generated by alternative splicing (McGraw K, et. al. PLoS One 2014). In a pilot study of Epo-refractory MDS patients, addition of EA yielded erythroid responses in 28% of patients who were unresponsive to LEN alone, suggesting that LEN may overcome resistance and augment response to rhEpo (Komrokji R, et. al. Blood 2012). To test this hypothesis, randomized phase III trial was performed comparing treatment with LEN to LEN+EA in LR non-del(5q) MDS patients who were refractory to, or not candidates for treatment with rhEpo.

Methods

Patients with Low or Intermediate-1 (Int-1) risk IPSS MDS with hemoglobin <9.5 g/dL who were unresponsive to rhEpo treatment or were TD (>2 units/mo) with serum Epo >500 mU/mL were eligible for study. Patients were stratified by serum Epo level and prior rhEpo (EA vs. darbepoetin vs. none) then randomized to treatment with LEN 10 mg/d×21d q4 wk (Arm A) or LEN+EA 60,000 U SC/wk (Arm B). Primary endpoint was IWG 2006 major erythroid response (MER) rate after 4 cycles. Arm A non-responders were offered cross-over to combined therapy. Secondary endpoints included analysis of response biomarkers.

Results

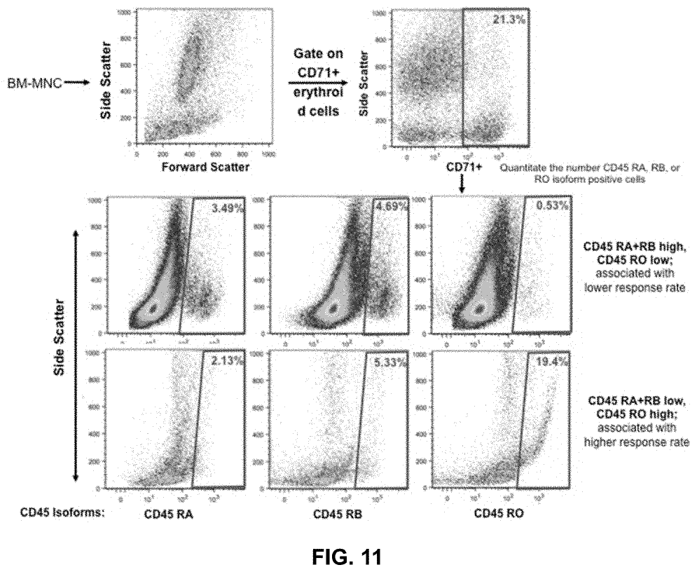

Patients were enrolled and 195 were randomized and are included in the primary analysis. Interim analysis of 163 patients (Arm A, 81; B, 82) showed that the study met predefined stopping criteria. Baseline characteristics were balanced between arms. Median age was 74 years (range, 47-89) receiving a median of 2 RBC units/mo (0-8). Overall, 64 (39%) patients had Low IPSS risk and 90 (55%) Int-1 risk. Among these, 150 received prior rhuEpo (92%) and 27, azanucleosides (17%). In an ITT analysis, MER rate was significantly higher with combination therapy, Arm B 25.6% (n=21) vs. Arm A 9.9% (n=8) (P=0.015). Among 116 patients evaluable at week 16, 33.3% (20/60) and 14.3% (8/56) achieved MER, respectively (P=0.018), with a median duration of response of 25.4 months vs. not reached in Arm A responders. Response to combined treatment was associated with baseline CD45-isoform distribution in erythroid precursors. As shown in FIG. 11, patients achieving MER had a significantly lower CD45 RA+RB:RO ratio (median, 1.51) compared to non-responders (median, 4.21; P=0.04), favoring homo-dimerization of the short CD45-RO isoform and inhibition of phosphatase activity. MER rate in Arm B patients with a low isoform ratio (<median) was 72.7% vs. 18.2% in the high ratio group (P=0.03). Thirty-four Arm A non-responders crossed over to combination-therapy with only 1 MER. There was no difference in the frequency or distribution of >Grade 3, non-hematologic AEs.

### CONCLUSIONS

LEN restores sensitivity to rhEpo in Epo-refractory LR-non-del(5q) MDS patients to yield durable and significantly higher rates of erythroid response to combination treatment without added toxicity. Erythroid CD45 isoform profile can serve as a response biomarker for selection of candidates for combination therapy.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

What is claimed is:

1. A method for treating a patient with a erythropoietin (Epo)-refractory, Lower Risk (LR) Non-deletion 5q [Del(5q)] myelodysplastic syndrome (MDS), comprising assaying CD71^{Hi} erythroid precursors from a biological sample from a patient in need thereof for a CD45 isoform profile, and treating the patient with a combination of lenalidomide and erythropoietin if the erythroid precursors have a predominance of small CD45 RO isoform compared to large CD45 RA and CD45 RB isoforms.

2. The method of claim 2, wherein the erythropoietin comprises recombinant erythropoietin.

3. The method of claim 1, wherein the biological sample is assayed for CD45 isoforms on CD71^{Hi} erythroid precursors by flow cytometry.

4. The method of claim 1, wherein the biological sample is assayed for gene expression of CD45 isoforms.

5. The method of claim 1, comprising treating the patient with a combination of lenalidomide and erythropoietin if the subject has a RA+RB:RO ratio less than 3.0.

6. The method of claim 1, wherein the lenalidomide and erythropoietin are co-administered in a single composition.

7. The method of claim 1, wherein the lenalidomide and erythropoietin are sequentially administered.

8. The method of claim 1, wherein the biological sample comprises bone marrow mononuclear cells (BM-MNC).

9. A method for predicting whether a patient with a erythropoietin (Epo)-refractory, Lower Risk (LR) Non-deletion 5q [Del(5q)] myelodysplastic syndrome (MDS) will respond to treatment with lenalidomide and erythropoietin, comprising assaying CD71^{Hi} erythroid precursors from a biological sample from a patient in need thereof for a CD45 isoform profile, wherein a predominance of small CD45 RO isoform compared to large CD45 RA and CD45 RB isoforms is an indication that the patient will respond to treatment with lenalidomide and erythropoietin.

10. The method of claim 9, wherein the biological sample is assayed for CD45 isoforms on CD71^{Hi} erythroid precursors by flow cytometry.

11. The method of claim 9, wherein the biological sample is assayed for gene expression of CD45 isoforms.

12. The method of claim 9, wherein the biological sample comprises bone marrow mononuclear cells (BM-MNC).
